SEQUENCE LISTING (4 sequences)

SEQ ID NO: 1 - DNA, 19 bp, Artificial Sequence
  primer for beta-actin
accctgaagtaccccatcg

SEQ ID NO: 2 - DNA, 17 bp, Artificial Sequence
  primer for beta-actin
cgtgatggactccggtg

SEQ ID NO: 3 - DNA, 21 bp, Artificial Sequence
  primer for MMP-1
aaaatcctgtccagcccatcg

SEQ ID NO: 4 - DNA, 22 bp, Artificial Sequence
  primer for MMP-1
ttctgtccctgaacagcccagt

(19) United States
(12) Patent Application Publication
(10) Pub. No.: US 2011/0064682 A1
(43) Pub. Date: Mar. 17, 2011

(54) COSMETIC COMPOSITION COMPRISING A STEM-CELL CULTURE FLUID, AND A PRODUCTION METHOD THEREFOR

(75) Inventor: Myeong Jin NAM, Seoul (KR)

(73) Assignees: Myeong Jin Nam, Seoul (KR); HAN CELL, Incheon (KR)

(21) Appl. No.: 12/941,359

(22) Filed: Nov. 8, 2010

Related U.S. Application Data
(63) Continuation-in-part of application No. PCT/KR2009/002410, filed on May 7, 2009.

(30) Foreign Application Priority Data
May 7, 2008 (KR) 10-2008-0042564

(51) Int. Cl. A61K 8/98; A61Q 19/02; A61Q 19/08; C12N 5/07; C12N 5/074

(52) U.S. Cl. 424/62; 424/93.7; 435/325; 435/366

(57) ABSTRACT

The present invention relates to a method of improving wrinkle, whitening or anti-oxidation of a skin, and more specifically, to a method of improving wrinkle, whitening or anti-oxidation of a skin, comprising applying a cosmetic composition comprising a stem cell culture fluid as an active ingredient to the skin. Further, the present invention provides a method for preparing the cosmetic composition, comprising the steps of culturing stem cells and isolating the stem cells from the culture fluid.

### CROSS-REFERENCE TO RELATED APPLICATIONS

This is a Continuation in Part of International patent application PCT/KR2009/002410, filed May 7, 2009, which claims the priority of Korean patent application KR 10-2008-0042564, filed May 7, 2008, each of which is herein incorporated by reference in its entirety.

### BACKGROUND OF THE INVENTION

1. Field of the Invention

The present invention relates to a method of improving wrinkle, whitening or anti-oxidation of a skin, comprising applying a cosmetic composition comprising a stem cell culture fluid as an active ingredient to the skin. Further, the present invention provides a method for preparing the cosmetic composition, comprising the steps of culturing stem cells and isolating the stem cells from the culture fluid.

2. Description of the Related Art

The process of skin aging can be divided into two occurring processes: one is ‘intrinsic aging’ attributable to passage of time, and the other is ‘extrinsic aging’ attributable to environmental factors such as ultraviolet (UV) irradiation or pollution. Until now, various theories of skin aging have been suggested, but the most scientific approach of them is the oxidation reaction theory. Human skin is composed of the epidermis and the dermis, and the connective tissue. Among them, stratum corneum is made up of dead cells formed through the proliferation of basal epidermal keratinocytes, and functions to protect the human body from the environment. In addition, the dermis, which supports the skin, is composed of collagen and elastin to provide the skin with elasticity, and thus functions to sustain the skin. The oxidation reaction theory is a theory, in which collagen and elastin are damaged due to free radicals produced by external factors such as UV radiation, leading to reduced elasticity and wrinkle formation.

Up to now, materials such as retinol or plant extracts have been used in order to prevent skin aging, in particular, wrinkles. However, these materials are disadvantageous in that they have weak effects or induce skin irritation, redness, or inflammation to generate adverse effects.

Generally, human skin color is determined hereditarily according to the concentration and distribution of melanin in the skin, but it can also be influenced by environmental or physiological conditions such as solar ultraviolet rays, fatigue, and stress. Melanin is produced through a non-enzymatic oxidation reaction after the enzyme tyrosinase acts on the amino acid tyrosine changing it into DOPA and dopaquinone. When the melanin synthesis excessively occurs inside the skin, the tone of skin darkens and chloasma and freckles can be generated. Accordingly, when the melanin synthesis inside the skin is inhibited, the skin becomes bright to realize the skin whitening, and hyperpigmentation such as chloasma or freckles due to ultraviolet rays, hormonic and genetic factors can be also improved.

Conventionally, materials having inhibitory activity against tyrosinase such as hydroquinone, ascorbic acid, kojic acid (Japanese Patent Publication No. 56-18569, Japanese Patent Kokai No. 53-3538), or glutathione have been used together with cosmetic or ointment for the purpose of skin whitening or improving chloasma or freckles. Although hydroquinone has been the most used skin lightener for decades, its long-term use may cause allergic reaction (white spot, black spot), and thus its use has been restricted in many countries. Further, thiol compounds such as glutathione or cysteine have unique unpleasant odors as well as problems in transdermal absorption. In addition, glycosides and derivatives thereof are problematic in that they cannot be appropriately used as mixed ingredients of cosmetics due to their high polarities. A skin-whitening composition for external use has been disclosed in the following patents: a skin-whitening cosmetic composition comprising an emblica extract and a Ramulus mori extract (Korea Patent No. 449345), a novel chroman derivative, a preparation method thereof, and a composition for external use comprising the same (Korea Patent No. 456974), and a skin-whitening composition comprising protocatechuic aldehyde as an active ingredient (Korea Patent No. 336180).

Further, ascorbic acid, which has an inhibitory activity on tyrosinase to show a whitening effect, is easily oxidized, so that cosmetics mixed with it have problems of color and odor changes. To overcome the instability, derivatives thereof, such as ascorbyl-palmitate, ascorbyl-dipalmitate, ascorbyl stearate, and ascorbyl magnesium phosphate, have been developed and used. However, these components are still disadvantageous in that they cannot easily penetrate the skin, if special techniques are not employed, and they have a low tyrosinase inhibitory activity. Accordingly, there is a need for the development of inhibitors, which exhibit a tyrosinase inhibitory activity even in a small amount and is capable of inhibiting melanin biosynthesis.

The present inventors have made an effort to develop a cosmetic material having an excellent wrinkle improving effect, whitening effect, and antioxidant effect. They found that a composition comprising the stem cell culture fluid is able to increase synthesis of collagen and elastin, reduce the expression of MMP-1 which degrades the above proteins, and suppress melanin synthesis in melanocytes, and also has a function of inactivating free radicals, and thus has excellent effects as a functional cosmetic material, thereby completing the present invention.

### SUMMARY OF THE INVENTION

It is an object of the present invention to provide a method of improving wrinkle, whitening or anti-oxidation of a skin, comprising applying a cosmetic composition comprising a stem cell culture fluid as an active ingredient to the skin.

It is another object of the present invention to provide a method for preparing the cosmetic composition, comprising the steps of culturing stem cells and isolating the stem cells from the culture fluid.

### BRIEF DESCRIPTION OF THE DRAWINGS

FIG. 1 is a graph showing collagen synthesis after treatment of human fibroblast with cord blood-derived stem cell culture fluid;

FIG. 2 is a graph showing elastin synthesis after treatment of human fibroblast with cord blood-derived stem cell culture fluid;

FIG. 3 is the result of RT-PCR showing inhibition of MMP-1 synthesis after treatment of human fibroblast with cord blood-derived stem cell culture fluid;

FIG. 4 is the result of Western blot showing inhibition of MMP-1 synthesis after treatment of human fibroblast with cord blood-derived stem cell culture fluid;

FIG. 5 is the result of measuring tyrosinase activity of melanoma cells after treatment of B16F1 melanoma cells with CM obtained by MSC culture and addition of 2 μM of α-MSH;

FIG. 6 is the result of measuring melanin synthesis of melanoma cells after treatment of B1F1 melanoma cells with CM obtained by MSC culture and addition of 2 μM of α-MSH;

FIG. 7 is the result of measuring the antioxidative enzyme SOD (superoxide dismutase) activity of HDF after addition of the concentrated CM obtained by MSC culture to HDF treated with H₂O₂ (1 mM); and

FIG. 8 is the result of measuring the antioxidative enzyme GPx (Glutathione Peroxidase) activity of HDF after addition of the concentrated CM obtained by MSC culture to HDF treated with H₂O₂ (1 mM).

### DESCRIPTION OF THE PREFERRED EMBODIMENTS

In one aspect, the present invention relates to a method of improving wrinkle, whitening or anti-oxidation of a skin, comprising applying a cosmetic composition comprising a stem cell culture fluid as an active ingredient to the skin.

As used herein, the term ‘stem cell culture fluid’ refers to a material that comprises the components contained in the media obtained by culturing stem cells, and the type of stem cells for preparation of the culture fluid is not limited. For example, the stem cells for preparation of the culture fluid may be embryonic stem cells or adult stem cells. Furthermore, adult stem cells may be derived from adult stem cells of all tissues. For example, adult stem cells may be selected from bone marrow-derived, cord blood-derived, blood-derived, liver-derived, skin-derived, stomach-derived, placenta-derived, nerve-derived, adrenal gland-derived, epithelium-derived, and uterus-derived adult stem cells, and preferably cord blood-derived adult stem cells. In the specific Example of the present invention, cord blood-derived adult stem cells were used to prepare the culture fluid.

The media for stem cell culture may be any base media known in the art without limitation. The base media may be artificially synthesized, or commercially available media. Examples of the commercially available media include DMEM (Dulbecco's Modified Eagle's Medium), MEM (Minimal Essential Medium), BME (Basal Medium Eagle), RPMI 1640, F-10, F-12, α-MEM (α-Minimal essential Medium), G-MEM (Glasgow's Minimal Essential Medium) and Isocove's Modified Dulbecco's Medium, but are not limited thereto, and also may be α-MEM.

Further, the base media preferably contains 0.1% to 20%, and more preferably 2% to 5% FBS (fetal bovine serum). In the specific Example of the present invention, the cord blood-derived adult stem cells were cultured in α-MEM, and 2% and 5% FBS were used to assess the anti-wrinkle, whitening and anti-oxidant effects.

As used herein, the term ‘improving wrinkle’ means to maintain or reinforce the ability that is related to skin wrinkle and elasticity. In the skin structure, collagen fiber (collagen) and elastic fiber (elastin) present in the dermis are major proteins responsible for skin elasticity, and collagen biosynthesis is affected by intrinsic or extrinsic factors. In particular, the intrinsic factor is natural aging, which causes a reduction in cellular activity and collagen fiber. The extrinsic factor is excessive ultraviolet irradiation and stress, which produce oxygen free radicals reactive with thiol (—SH) group of protein, leading to inhibition of enzymatic activity or an increase in the expression of collagenase (Matrix Metalloproteinases-1: MMP-1) responsible for degradation of collagen and elastin, resulting in wrinkle formation and reduced elasticity.

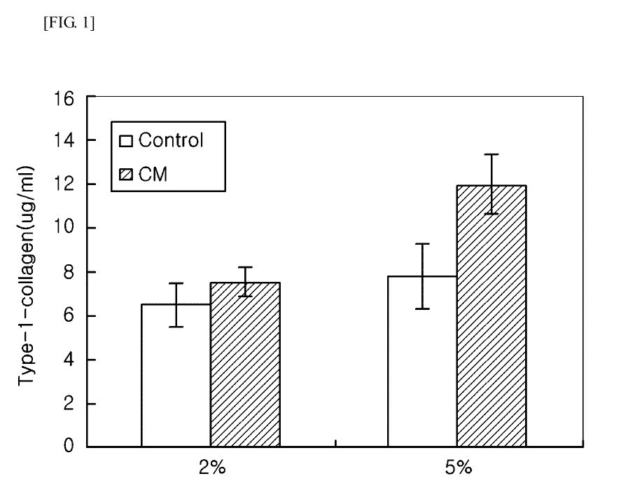

Accordingly, in connection with wrinkle improvement, the present inventors demonstrated in the specific Example that biosynthesis of collagen and elastin was increased, when fibroblast was cultured in the stem cell culture fluid. They also confirmed that the expression of MMP-1 responsible for degradation of collagen and elastin was inhibited (FIGS. 1 to 4).

As used herein, the term ‘whitening’ means to inhibit biosynthesis of melanin pigment in the melanocytes that determine the skin color, so that the skin darkening is prevented. The stem cell culture fluid of the present invention is a culture fluid that particularly performs the whitening effect. Preferably, the composition comprising the stem cell culture fluid of the present invention has an ability of inhibiting biosynthesis of melanin pigment in the melanocytes that determine the skin whitening. More preferably, the culture fluid having the ability of inhibiting biosynthesis may be a composition comprising the cord blood-derived stem cell culture fluid.

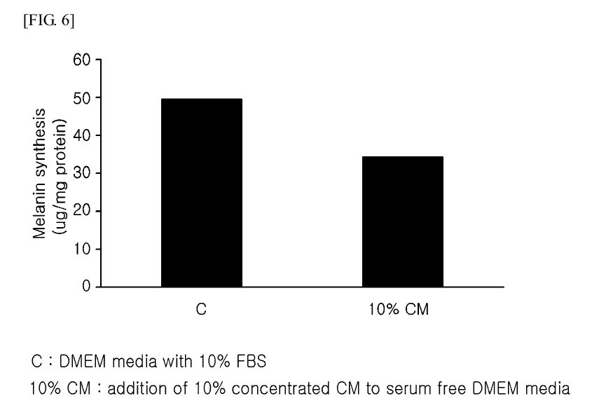

Melanocytes are cells located in the bottom layer (the stratum basale) of the skin's epidermis and in the middle layer of the eye (the uvea). Through a process called melanogenesis, these cells produce melanin, which is a pigment found in the skin, eyes, and hair. Generally, around 1000 to 2000 melanocytes are found in 1 mm² of skin, and melanocytes occupy 5 to 10% of the basal layer of the epidermis. The melanocytes used in the experiment may be any one of CCL-49 (RPMI 1846), CCL-53.1 (Clone M-3 [Cloudman S91 melanoma]), CRL-11147 (A2058), CRL-7572 (Hs 839.T), CRL-6322 (B16-F0), CRL-6323 (B16-F1), CRL-1974 (COLO 829), and CRL-1585 (C32) of ATCC (American Type Culture collection), and are not limited to melanocytes under particular conditions. In the specific Example of the present invention, fibroblasts were extracted from the experimental group with the stem cell culture fluid and the control group without the stem cell culture fluid, and then absorbance of melanin was measured and quantified, thereby assessing the melanin biosynthesis (FIG. 6).

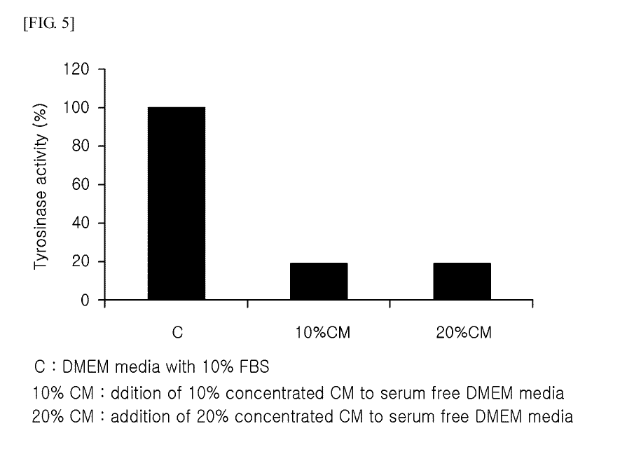

Meanwhile, tyrosinase present in melanocytes catalyzes the conversion of tyrosine to dopa and dopaquinone, which then produce melanin via dopachrome. Tyrosinase is required for melanocytes to produce melanin from the amino acid tyrosine. Melanin present in the skin functions to protect the body from UV. However, it has been known that excessive production of melanin causes formation of chloasma or freckles, accelerates skin aging, and causes skin cancer. Thus, recent studies have been actively made to develop agents for the prevention of excessive production of melanin. In the specific Example of the present invention, fibroblasts were extracted from the experimental group with the stem cell culture fluid and the control group without the stem cell culture fluid, and disrupted, and then tyrosinase was isolated therefrom. The absorbance was measured using a microplate reader, thereby assessing the inhibitory activity on tyrosinase (FIG. 5).

As used herein, the term ‘anti-oxidation’ effect means a function to inhibit oxidation of skin components, which is caused by highly reactive oxygen and free radical produced by UV. The reactive oxygen and free radical oxidize skin components to produce peroxide, which causes structural and functional damages to the skin, leading to acceleration of aging. The anti-oxidant effect of the present invention performs a function of protecting the skin therefrom. To achieve the objects of the present invention, the composition comprising a stem cell culture fluid functions to inactivate free radicals, and preferably, the composition is able to remove superoxide radicals that are produced by xanthine-xanthine oxidase system.

The antioxidant can be largely divided into primary antioxidant, secondary antioxidant, and tertiary antioxidant. A primary antioxidant plays a role in preventing the production of new reactive oxygen radicals in the body. A secondary antioxidant plays a role in scavenging reactive oxygen radicals, and preventing chain reaction. A tertiary antioxidant plays a role in recovering biological molecules damaged by reactive oxygen radicals. Examples of the primary antioxidant include SOD (SuperOxide Dismutase), GPx (Glutathione Peroxidase), metal binding proteins (ferritin, caeruloplasmin etc), selenium and GR (Glutathione Reductase), and examples of the secondary antioxidant include Vitamin E (α-tocopherol), Vitamin C (ascorbate), β-carotene (Provitamin A) and uric acid, bilirubin, and albumin, DNA repair enzyme and methionine superoxide reductase. The media composition of the present invention is able to improve the activity of such antioxidants, and preferably, the activity of the primary antioxidant. In the preferred Example of the present invention, it was confirmed that the activity of the primary antioxidant, SOD and GPx was increased. SOD is able to convert O₂ into H₂O₂, and GPx functions to convert H₂O₂ and lipid peroxide into non-toxic molecules before formation of reactive oxygen radicals. Preferably, it was confirmed that the composition comprising the stem cell culture fluid of the present invention improves the activity of SOD and GPx, thereby showing powerful antioxidant effects.

In the specific Example of the present invention, reactive oxygen radicals that are produced by xanthine-xanthine oxidase system are reacted with Nitroblue tetrazolium, and the color development was measured, followed by analysis of the radical scavenging effect. As a result, it was confirmed that the composition comprising the stem cell culture fluid has the antioxidant effects.

As used herein, the term ‘cosmetic composition’ refers to a composition comprising the stem cell culture fluid, and may be provided in any formulation. Examples of the formulation may include cosmetics prepared using the cosmetic composition, such as cream, pack, lotion, essence, skin lotion, foundation, and make-up base. In order to achieve the objects of the present invention, any of them may be prepared for commercialization, and the formulation is not limited to the above examples.

In another aspect, the present invention relates to a method for preparing the cosmetic composition, comprising the steps of culturing stem cells and isolating the stem cells from the culture fluid.

The preparation of the cosmetic composition may be performed by any method typically used. Preferably, a preparation method, comprising the steps of culturing stem cells; and isolating the stem cells from the culture fluid, is provided. The steps of culturing and isolating the stem cells may be performed by a typical method known in the art.

Hereinafter, the present invention will be described in detail in light of Examples. The present invention may, however, be embodied in many different forms by the typical method known in the art, and should not be construed as being limited to the Examples set forth herein.

### Example 1

### Preparation of Cord Blood-Derived Adult Stem Cell Culture Fluid

Cord blood-derived adult stem cells in passages 3 to 8 were used. The cells were aliquoted into a 100 mm-dish at a density of 4×10⁵, and after 24 hrs, replaced with 5 ml of 2% and 5% MEM media. To obtain secretory proteins produced by adult stem cells, the cells were cultured to approximately 90% confluency (sample group, Conditioned Media (CM)). About 5-6 days were required for the culture. As a control group, no cord blood-derived adult stem cells were aliquoted into a 100 mm-dish with 2% and 5% media (Control (C)) (hereinbelow, referred to as ‘sample group’ and ‘control group’).

### Example 2

### Stimulatory Effect of Adult Stem Cell Culture Fluid on Collagen Synthesis

Collagen is a biomaterial occupying most of the connective tissue, and responsible for skin elasticity. The body's production of collagen slows dramatically with aging. Therefore, to examine the effect of stem cell culture fluid of the present invention on the skin elasticity, the collagen synthesis was measured. First, human dermal fibroblasts were treated with the control group and the sample group prepared in Example 1 for 6 days, and then the amount of elastin in the sample was measured. 1 ml of Sircol dye reagent was added to the control group and the sample group, and then mixed using a shaker for 30 min. Subsequently, centrifugation was performed at 10,000×g for 10 min During this process, collagen-dye complex was precipitated and unbound dye remained in the supernatant. After centrifugation, the supernatant was removed, and 1 ml of alkali reagent was added to the collagen-dye complex pellet, and dye binding with collagen was dissolved by tapping the bottom of tube for 5-10 min. The fully dissolved alkali dye solution was measured at 540 nm, and then collagen synthesis was assessed.

The collagen synthesis under the conditions of 2% and 5% FBS of Example 1 was measured. Consequently, as shown in FIG. 1, collagen synthesis was increased under the condition of 2% FBS, but a significant increase was not observed. Under the condition of 5% FBS, approximately 35% significant increase was observed in the sample group, compared to the control group (FIG. 1).

### Example 3

### Stimulatory Effect of Adult Stem Cell Culture Fluid on Elastin Synthesis

Skin elasticity is determined by the elastic fibers present in the dermis, and the elastic fibers exist together with collagen fibers. Skin elasticity is maintained under the sufficient level of elastin and collagen. Human dermal fibroblasts were treated with the control group and the sample group prepared in Example 1 for 6 days, and then the amount of collagen in the media was measured. 1 ml of cold Fastin Precipitating reagent was added to the control group and the sample group, and reacted in an ice/water mixture in a refrigerator overnight. Next day, more ice was added thereto, and then left in the refrigerator for about 300 min. After 30 min reaction, elastin was precipitated at 10,000×g or higher for 10 min in the cold condition. After centrifugation, the supernatant was removed. 1 ml of Fastin Dye reagent and 100 ul of Elastin-dye complexing reagent were added to each tube, and then elastin pellet was mixed well by vortexing, and reacted using a shaker for 60 min. Then, centrifugation was performed at 10,000×g for 10 min to isolate the elastin-dye complex. After removing the supernatant, 1 ml of Fastin Dissociation reagent was added, and then the dye binding to elastin was dissolved by vortexing. Absorbance was measured at 513 nm, and then elastin synthesis was assessed.

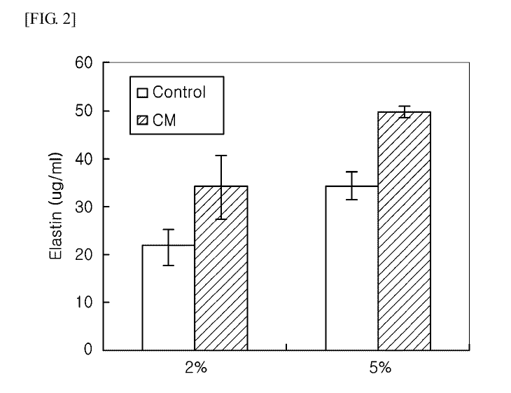

The elastin synthesis under the conditions of 2% and 5% FBS of Example 1 was measured. Consequently, as shown in FIG. 2, elastin synthesis was found to increase under both conditions. Under the condition of 5% FBS, a higher significance was found (FIG. 2).

### Example 4

### Inhibitory Effect on MMP-1 Expression

In vivo synthesis and degradation of extracellular matrix are coordinately balanced. With aging, however, the synthesis is reduced, and the expression of the collagen-degrading enzyme, matrix metalloproteinase (MMP), in particular, MMP-1 is promoted, leading to the reduced skin elasticity and wrinkle formation. The inhibitory effect on MMP-1 expression was confirmed by ELISA, RT-PCR, and Western blot analysis, as follows.

4-1. Analysis of Inhibitory Effect on MMP-1 Expression by ELISA Human fibroblast was inoculated into a 48-well plate at a density of 4×10⁵, and cultured in MEM media for 24 hrs. Subsequently, the media were replaced with the control group and the sample group, and cultured for another 6 days. Thereafter, MMP-1 was measured in accordance with ELISA. Cell debris was removed by centrifugation, and the supernatant was used for the experiment. The supernatant was treated with a primary antibody (MMP-1 monoclonal antibody), and incubated at 37° C. for 90 min Blocking was performed by treatment of 3% BSA for 1 hr. After treatment of secondary antibody for 90 min, a substrate solution was added, and incubated at room temperature for 30 min Absorbance was measured at 405 nm, and those of the control group and the sample group were compared with each other.

Consequently, as shown in the following Table 1, MMP-1 activity was remarkably inhibited from 174.7 ng/ml to 141.9 ng/ml under the condition of 2% FBS, and from 123.2 ng/ml to 27.4 ng/ml under the condition of 5% FBS, after treatment of the sample group (Table 1).

TABLE 1
Comparison of inhibitory effect on MMP-1 production by ELISA

| | | |
|---|---|---|
| 2% FBS | Control | 174.7 ng/ml |
| | CM | 141.9 ng/ml |
| 5% FBS | Control | 123.2 ng/ml |
| | CM | 27.4 ng/ml |

4-2. Analysis of Inhibitory Effect on MMP-1 Expression by RT-PCR

Human fibroblast was inoculated into a 100 mm dish at a density of 1×10⁶. After the cells were cultured to approximately 30% confluency, the media were replaced with the control group and the sample group, and cultured for another 6 days. Then, RNA was isolated from the cells by Guanidium thiocyanate-phenol-chloroform extraction using a Trizol reagent. 4 μg of RNA was subjected to RT-PCR using a RT-PCR (Reverse Transcriptase PCR) kit. For quantitation, beta-actin was used, and the PCR product was electrophoresed on a 1% agarose gel to confirm the result. The primers used in the experiment are as follows.

| | |
|---|---|
| Beta-actin | |
| 5′-accctgaagtaccccatcg-3′ | (SEQ ID NO. 1) |
| 5′-cgtgatggactccggtg-3′ | (SEQ ID NO. 2) |
| MMP-1 | |
| 5′-aaaatcctgtccagcccatcg-3′ | (SEQ ID NO. 3) |
| 5′-ttctgtccctgaacagcccagt-3′ | (SEQ ID NO. 4) |

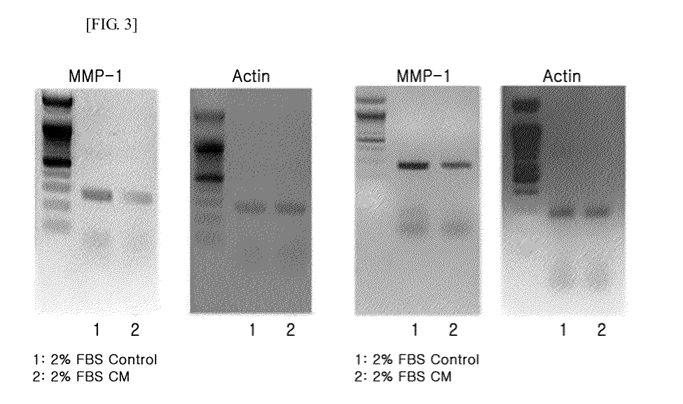

As a result, it was found that the MMP-1 expression was inhibited under the conditions of 2% and 5% FBS after treatment of the sample group, and the quantitation was performed using beta-actin (FIG. 3).

4-3. Analysis of Inhibitory Effect on MMP-1 Expression by Western Blotting

Human fibroblast was inoculated into a 100 mm dish at a density of 1×10⁶. After the cells were cultured to approximately 50% confluency, the media were replaced with the control group and the sample group, and cultured for another 6 days. Then, the cells were collected and disrupted, and then proteins were quantified. An equal amount of protein was loaded on a 10% polyacrylamide gel, followed by electrophoresis until the bands were separated. After electrophoresis, the gel was blotted on a nitrocellulose membrane. The membrane was blocked using 5% skim milk for 2 hrs, and washed with TBST three times, followed by reaction with MMP-1 monoclonal antibody at room temperature for 2 hrs. After washing with TBST three times, anti-mouse secondary antibody was reacted at room temperature for 1 hr, and the results of the control group and the sample group were compared with each other by western blot detection system.

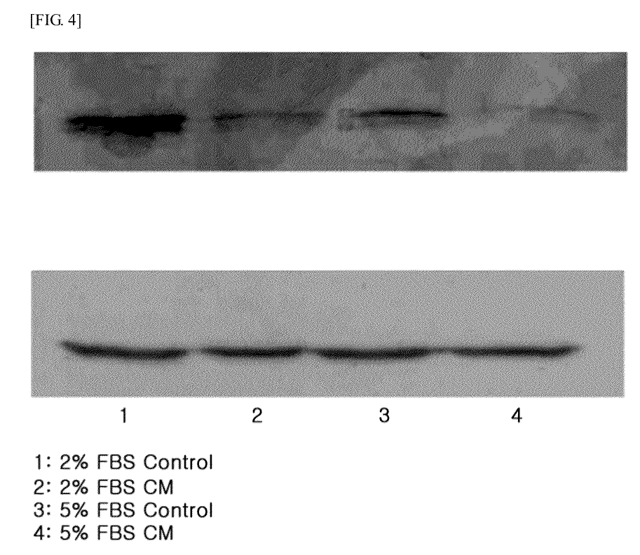

As a result, it was found that the MMP-1 expression was inhibited under the conditions of 2% and 5% FBS after treatment of the sample group, and the quantitation was performed using beta-actin (FIG. 4).

### Example 5

### Inhibition of Cellular Tyrosinase Activity in B16F1 Melanocyte

In this Example, inhibition of cellular tyrosinase activity in B16F1 melanocytes was assessed in order to confirm the whitening effect of the control group and the sample group obtained in Example 1. This cell is a mouse-derived cell, and synthesizes tyrosinase enzyme. This cell was obtained from ATCC (American Type Culture collection, 6323). Upon culturing the cells, the above samples were treated thereto, and then tyrosinase was isolated from the cells. Subsequently, the enzymatic activity was measured to compare the inhibition of tyrosinase activity.

B16F1 cells were inoculated into a 6-well plate at a density of 4×10⁵, and attached thereto. Then, the cells were treated with the control group and the sample group of Example 1, and cultured for another 6 days. After 6 days, the cells were detached by trypsin-EDTA treatment, and the number of cells was counted, followed by centrifugation for recovery. The cells were washed with PBS once, and 1 ml of homogenization buffer (50 mM sodium phosphate, pH 6.8, 1T Triton X-100, 2 mM PMSF) was added and vortexed for 5 min to disrupt the cells. Centrifugation was performed to recover the supernatant. 1.5 mM L-tyrosine, 0.06 mM L-DOPA, and the supernatant were added to the 50 mM phosphate buffer, and incubated at 37° C. for 30 min. The absorbance was measured at 490 nm using a microplate reader, thereby assessing the inhibitory activity on tyrosinase.

CM obtained from the MSC culture was treated to the melanocytes, and 2 μM of α-MSH was added thereto, and then tyrosinase activity of melanocytes was examined. As a result, it was found that the tyrosinase activity was remarkably inhibited by CM (FIG. 5).

### Example 6

### Inhibition of Melanin Production in B16F1 Melanocyte

In this Example, inhibition of melanin production in B16F1 melanocytes was assessed in order to confirm the whitening effect of the control group and the sample group obtained in Example 1.

This cell is a mouse-derived cell, and secretes the pigment melanin. B16F1 cells were inoculated into a 6-well plate at a density of 4×10⁵, and attached thereto. Then, the cells were treated with the control group and the sample group of Example 1, and cultured for another 6 days. After 6 days, the cells were detached by trypsin-EDTA treatment, and the number of cells was counted, followed by centrifugation for recovery. The cells were washed with PBS once, and 1 ml of homogenization buffer (50 mM sodium phosphate, pH 6.8, 1T Triton X-100, 2 mM PMSF) was added and vortexed for 5 min to disrupt the cells. 1 N NaOH (10% DMSO) was added to the supernatant obtained by centrifugation, and the extracted melanin was dissolved. Then, the absorbance of melanin was measured at 405 nm using a microplate reader, and the amount of melanin was quantified, thereby assessing the inhibition rate (%) of the sample on melanin production.

CM obtained from the MSC culture was treated to the melanocytes, and 2 μM of α-MSH was added thereto, and then melanin production in the melanocytes was examined. As a result, it was found that the melanin production was reduced by CM media (FIG. 6).

### Example 7

### Analysis on Anti-Oxidant Effect by Reactive Oxygen Radicals

Photoaging mechanism caused by ultraviolet rays is mediated via a free radical pathway. Free radicals are known to cause the destruction of connective tissue such as skin collagen, inhibit the function of cell membrane, accelerate DNA mutation, and induce the modification of the molecules involved in metabolism. The concept that free radicals are involved in the aging process means that antioxidants showing the effect of inactivating free radicals can impact the aging process. In this Example, the effect of scavenging superoxide radicals produced by xanthine-xanthine oxidase system was measured. The superoxide radicals produced by the reaction are reacted with nitroblue tetrazolium, resulting in blue color development. Thus, as superoxide radicals are scavenged, strength of blue color changes. 3×10⁻³ M xanthine, 3×10⁻⁴ M EDTA, 7.5×10⁻⁴ M NBT, and 0.15 mg/ml of BSA solution were added to the control group and the sample group, and mixed with each other, and then reacted at room temperature for 10 min. Thereafter, xanthine oxidase (0.25 U/ml) solution was added thereto, and left at room temperature for 20 min, and then absorbance was measured at 565 nm. Oxygen free radical scavenging capacity was calculated by the following formula: “Oxygen free radical scavenging capacity (%)=[(Absorbance of control group−Absorbance of sample)/Absorbance of control group]×100”.

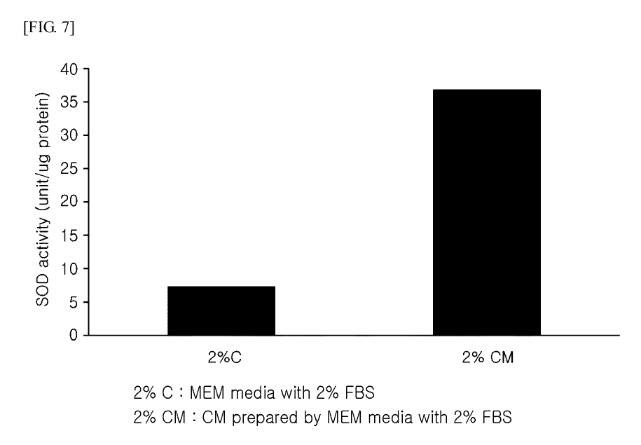

Oxygen free radicals were measured by activities of SOD (superoxide dismutase) and GPx (Glutathione Peroxidase), and SOD activity was examined by adding the concentrated CM obtained by MSC culture to HDF treated with 1 mM of H₂O₂, and measuring the antioxidative enzyme SOD activity of HDF. As a result, addition of 2% CM showed higher SOD activity than the control group (FIG. 7).

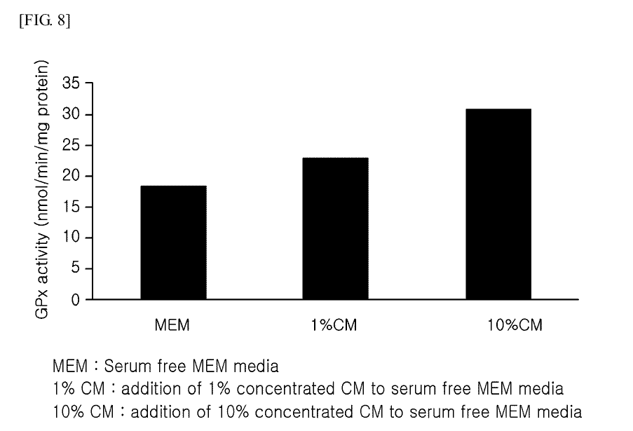

Further, these antioxidant effects can be examined by activity of GPx (Glutathione Peroxidase), in which the concentrated CM obtained by MSC culture was added to HDF treated with 1 mM of H₂O₂, and then the antioxidative enzyme activity of HDF was measured. As a result, addition of concentrated 10% CM showed higher GPx activity (FIG. 8).

### EFFECTS OF THE INVENTION

The stem cell culture fluid of the present invention has excellent anti-wrinkle and whitening effects, thereby showing excellent effects as a functional cosmetic material and composition.

What is claimed is:

1. A method of improving wrinkle, whitening or anti-oxidation of a skin, comprising applying a cosmetic composition comprising a stem cell culture fluid as an active ingredient to the skin.

2. The method according to claim 1, wherein the cosmetic composition comprises a stem cell culture fluid containing 0.1% to 20% FBS as an active ingredient.

3. The method according to claim 1, wherein the cosmetic composition comprises a stem cell culture fluid containing 2% to 5% FBS as an active ingredient.

4. The method according to claim 1, wherein the stem cell is one or more stem cells selected from the group consisting of bone marrow-derived, cord blood-derived, blood-derived, liver-derived, skin-derived, stomach-derived, placenta-derived, nerve-derived, adrenal gland-derived, epithelium-derived, and uterus-derived human adult stem cells, and embryonic stem cells.

5. The method of improving wrinkle of a skin according to claim 1, wherein the cosmetic composition has an activity of collagen synthesis.

6. The method of improving wrinkle of a skin according to claim 1, wherein the cosmetic composition has an activity of elastin synthesis.

7. The method of improving wrinkle of a skin according to claim 1, wherein the cosmetic composition has an inhibitory activity on MMP (matrix metalloproteinase)-1 expression.

8. The method of whitening of a skin according to claim 1, wherein the cosmetic composition has an inhibitory activity on tyrosinase activity in melanocytes.

9. The method of whitening of a skin according to claim 1, wherein the cosmetic composition has an inhibitory activity on melanin synthesis in melanocytes.

10. The method of whitening of a skin according to claim 8, wherein the melanocyte is B16F1.

11. The method of whitening of a skin according to claim 9, wherein the melanocyte is B16F1.

12. The method of anti-oxidation of a skin according to claim 1, wherein the cosmetic composition has an activity of scavenging reactive oxygen radicals produced by xanthine-xanthine oxidase system.

13. The method of anti-oxidation of a skin according to claim 12, wherein the activity of scavenging reactive oxygen radicals is obtained by increased activity of SOD (SuperOxide Dismutase) or GPx (Glutathione Peroxidase).

14. A method for preparing the cosmetic composition comprising a stem cell culture fluid as an active ingredient, comprising the steps of:
a) culturing stem cells; and
b) isolating the stem cells from a culture fluid.

15. The method according to claim 14, wherein the cosmetic composition comprises a stem cell culture fluid containing 0.1% to 20% FBS as an active ingredient.

16. The method according to claim 14, wherein the cosmetic composition comprises a stem cell culture fluid containing 2% to 5% FBS as an active ingredient.

17. The method according to claim 14, wherein the stem cell is one or more stem cells selected from the group consisting of bone marrow-derived, cord blood-derived, blood-derived, liver-derived, skin-derived, stomach-derived, placenta-derived, nerve-derived, adrenal gland-derived, epithelium-derived, and uterus-derived human adult stem cells, and embryonic stem cells.

18. The method according to claim 14, wherein the cosmetic composition has an activity of collagen synthesis, an activity of elastin synthesis or an inhibitory activity on MMP (matrix metalloproteinase)-1 expression.

19. The method according to claim 14, wherein the cosmetic composition has an inhibitory activity on tyrosinase activity in melanocytes or an inhibitory activity on melanin synthesis in melanocytes.

20. The method according to claim 19, wherein the melanocyte is B16F1.
